(19) United States
(12) Patent Application Publication
(10) Pub. No.: US 2026/0124558 A1
(43) Pub. Date: May 7, 2026

(54) DEVICE FOR RECOVERING MEDICAL RADIOACTIVE ISOTOPES

(71) Applicant: Loïc DJAILEB, Saint-Nazaire-Les-Eymes (FR)

(72) Inventor: Loïc DJAILEB, Saint-Nazaire-Les-Eymes (FR)

(21) Appl. No.: 19/115,891

(22) PCT Filed: Sep. 29, 2023
(86) PCT No.: PCT/FR2023/051513

§ 371 Date: Mar. 27, 2025

(30) Foreign Application Priority Data
Sep. 30, 2022 (FR) FR2210010

(51) Int. Cl. B01D 15/36; A61B 6/40; G01N 33/493

(52) U.S. Cl. CPC B01D 15/362; G01N 33/493; A61B 6/4057

(57) ABSTRACT

A device for recovering a radioactive isotope of interest present in the urine of a patient, intended to be connected to toilets. The device includes a urine collector, a treatment unit, a pressure management system. The treatment unit includes a filtration and recovery device for each radioactive isotope of interest configured to filter and recover the radioactive isotope of interest, a sorting module connected to the urine collector configured to identify the radioactive isotope of interest in the collected urine and direct the collected urine to the filtration and recovery device corresponding to the detected radioactive isotope, and at least one primary storage chamber in fluidic connection with the filtration and recovery device configured to recover the corresponding radioactive isotope.

### FIELD OF THE INVENTION

The present invention relates to devices that make it possible to recover radioactive isotopes from biological liquids.

### PRIOR ART

In a manner known per se, nuclear medicine is the medical specialty using unsealed radioactive sources for diagnostic and therapeutic purposes.

More particularly, internal vectorised radiation therapy (IVR) is the therapeutic branch of nuclear medicine. The development thereof has been rapid in recent years, particularly in the context of the management of metastatic prostate cancer with so-called “¹⁷⁷Lu-PSMA” treatments targeting PSMA, prostate specific membrane antigens (PSMA) expressed by cancer cells.

During treatment with IVR, particularly with ¹⁷⁷Lu-PSMA, most of the treatment is injected and eliminated by urine. In the current state of the art, this radioactivity is thereafter stored and then eliminated in nature when the regulatory threshold is reached. More precisely, currently in nuclear medicine departments, the management of liquid radioactive waste requires the installation of expensive furniture and logistics with the transport and storage of radioactivity in decay tanks.

All of the elements currently used relate to:
radiation-protected toilets,
lead-sealed devices for the radiation protection of personnel,
radioactive decay tanks,
the systems for managing the concentrations of radioactive isotope(s) in aqueous solutions (non-selective).

Before being disposed of in nature, liquid radioactive waste is stored and transported in decay tanks.

The recent increase in indications for use of IVR, particularly in metastatic prostate cancer with ¹⁷⁷Lu-PSMA, leads to an increased need for production of ¹⁷⁷Lu (radioactive isotope used for this indication). In general, and beyond the example of prostate cancer cases, projections envisage an increase in IVR indications in the coming years with ¹⁷⁷Lu but also with other radioactive isotopes (β, α emitters, etc.).

The use of IVR in the coming years must meet major challenges related to the increase in indications. These challenges correspond schematically to:
increasing production of radioactive isotopes,
better control of the elimination of these radioactive isotopes, currently eliminated in nature.
logistical adaptability for increasing patient reception.

The radioactive waste generated by the patient has a half-life of less than 100 days and is subsequently eliminated in nature when the radioactivity is less than 10 becquerels per litre. This may generate, in the event of a significant increase in the number of patients to be treated, problems following saturation of the radioactive tanks related to the increasing number of patients treated.

Currently known techniques make radiation protection possible for health personnel before elimination in nature, in compliance with the regulations in force. However, these techniques do not make it possible to:
selectively isolate radioactive isotopes,
isolate radioactive isotopes for the reintegration thereof into a GMP (good manufacturing practice) production line,
create autonomous therapeutic units for the management thereof of radioactive waste and independent of a common radioactivity collection circuit (connected radioactive tank).
integrate these units into a radiopharmaceutical drug production circuit.

Therefore, there is currently a need for a technical solution to the above-mentioned list of problems. The present invention thus seeks to remedy all of these shortcomings.

The object of the present invention is particularly to propose, on the one hand, a more economical solution for limiting both the production and storage of the necessary radioactive isotopes and, on the other hand, an environmental protection solution for limiting the pollution thereof by avoiding the discharge of said radioactive isotopes into nature. Another object of the present invention is to offer to care units the possibility of quickly adapting the need for protected room installation to the clinical need.

### SUMMARY

This objective is achieved, in accordance with the invention, thanks to a device for recovering at least one radioactive isotope of interest present in the urine of a patient, the device being intended to be connected to toilets, the device comprising a urine collector, a treatment unit, a pressure management system configured to move the urine from the urine collector to and within the treatment unit, the treatment unit including:
at least one filtration and recovery device specific to each radioactive isotope of interest, including an ion retention element and at least one reactive solution reservoir, the filtration and recovery device being configured to filter and recover the radioactive isotope of interest,
a sorting module connected to the urine collector configured to identify the radioactive isotope of interest in the collected urine and direct the collected urine to the at least one filtration and recovery device corresponding to the detected radioactive isotope,
at least one primary storage chamber in fluidic connection with each filtration and recovery device configured to recover the corresponding radioactive isotope.

Thus, this solution makes it possible to achieve the aforementioned objective. In particular, the present innovation makes it possible to recycle radioactive isotopes eliminated by the urine of the patients. Given that the published data make it possible to estimate an elimination of Lu-PSMA at about 45% at 6 hours of injection, this recovery is therefore significant and makes it possible to respond to the environmental and economic problems mentioned above by:
a reduction in the production needs of the various radioactive isotopes,
a solution to the prolonged storage of radioactive isotopes as well as a reduction in the elimination thereof in uncontrolled environments.

The device for recovering radioactive isotopes according to the invention may comprise one or more of the following features, considered separately from one another or in combination with one another:
the sorting module may be provided with a spectrometer configured to detect the radioactive isotope of interest,
the device may include a primary storage chamber for each radioactive isotope of interest,
the suction of the urine of the patient can be activated by detecting fluid in the urine collector,
the device may comprise a secondary storage chamber configured to recover aqueous elements of the collected urine, said secondary storage chamber being in fluidic connection with the sorting module and each filtration and recovery device,
the pressure management system may be configured to generate:
   a negative pressure in the sorting module in response to the detection of liquid in the urine collector,
   a positive pressure in the sorting module in response to the detection of the radioactive isotope of interest.
   a negative pressure in the primary storage chamber in response to a release of the reactive solution from the reservoir of the filtration and recovery device,
the ion retention element of the filtration and recovery device may be a cation exchange resin,
the device may also include a stool collector,
the device may also include a purification chamber,
the purification chamber may include a dissociation module.

Another object of the present application relates to a method for recovering at least one radioactive isotope of interest present in the urine of a patient, implemented by means of the device as described above. The method includes the following steps of:
collecting the urine of the patient by means of the urine collector,
sucking the urine to the sorting module,
detecting the radioactive isotope of interest and the radioactive isotope of interest to a corresponding filtration and recovery device,
trapping the radioactive isotope of interest on the retention device of the filtration and recovery device,
releasing the reactive solution in such a way as to release the radioactive isotope of interest,
fluidically transferring and storing said radioactive isotope of interest to/in the primary storage chamber.

### BRIEF DESCRIPTION OF THE FIGURES

The invention will be better understood, and other aims, details, features and advantages thereof will become more apparent upon reading the following detailed description of embodiments of the invention, given purely by way of illustrative and non-limiting examples, with reference to the appended schematic drawings. On these drawings:

FIG. 1 is a general block diagram of the collection device according to a first embodiment

FIG. 2 is a general block diagram of the collection device according to a second embodiment

FIG. 3 is a block diagram of a filtration and recovery device and a purification device according to the present invention.

### DETAILED DESCRIPTION

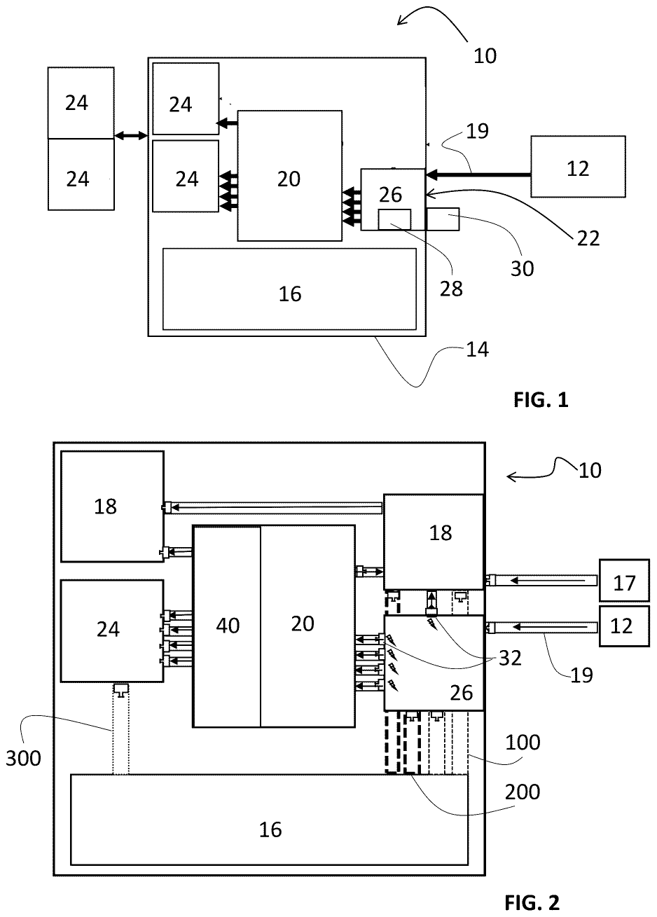

As can be seen in FIG. 1, the present invention relates to a device 10 for recovering at least one radioactive isotope of interest present in the urine of a patient.

The device 10 is intended to be connected to toilets whereon the patient sits to relieve themselves, after a medical intervention involving a radioactive isotope of interest. The device 10 has the proportions of a cabinet, preferably of a maximum of 2 m², in such a way as to be able to be integrated into the infrastructure of a room.

The device 10 according to the present invention comprises, for this purpose:
a urine collector 12,
a treatment unit 14,
a pressure management system 16 configured to move urine from the collector 12 to and within the treatment unit 14.

The urine collector 12 has a general bowl shape adaptable to both radiation-protected and non-radiation-protected toilets. The urine collector 12 is at least partially made of a non-adherent material for eliminating all of the collected elements, such as for example Teflon.

In order to put the urine collector 12 in fluidic contact with the treatment unit 14, the bottom of the urine collector 12 has a valve that opens by suction.

This suction is triggered by the pressure management system 16, when liquid is detected in the bottom of the urine cup 12. This detection occurs more particularly by detecting a negative pressure between 0.5 and 1 bar. The detection may also correspond to the presence of the patient on the toilet. This detection is made possible particularly by the presence of sensors on the urine collector 12. In this step, the pressure management system 16 plays a role of urine suction system. This suction makes it possible to avoid the stagnation of radioactive urine in the urine collector 12 outside of the treatment unit 14.

The rinsing of the urine collector 12 is preferably managed automatically by a water distribution device. This rinsing is preferably minimal and is carried out by a plurality of water jets in order to limit the amount of water added while eliminating the radioactivity possibly residual in the urine collector 12.

In a series of alternative embodiments, the device has two different collectors 12, 17: the urine collector 12 and a stool collector 17. The two collectors 12, 17 are separate and fit on existing radiation-protected toilets, that is to say the toilets have a central separator with the urine collector 12 at the front and the stool collector 17 at the rear.

In a first alternative embodiment, the stool collector 17 is not connected to the treatment unit 14. The stool collector 17 is in fluidic connection with a secondary storage chamber 18.

In order to put the stool collector 17 in fluidic contact with the secondary storage chamber 18, the bottom of the stool collector 17 has a flap that opens by suction. This suction is triggered in a similar way as described above for the urine collector 12. The rinsing of the stool collector 17 is also similar to that of the urine collector 12 described above.

The secondary storage chamber 18 is preferably in the form of a radiation-protected mini-tank. The secondary storage chamber 18 includes a motorised propeller for crushing the collected stools. The secondary storage chamber 18 also includes an element or a product for stopping the fermentation of stools (e.g. lime). It also makes it possible to implement a method for liquefying stools from liquids eliminated by the treatment unit 14 and from a rinsing liquid of the stool collector 17.

The urine collector 12 is connected to the treatment unit by an inlet pipe 19 made at least partially of a radiation-protective material. This inlet pipe 19 is configured to make the distance travelled by the urine between the urine collector 12 and the treatment unit 14 as short as possible. This inlet pipe 19 has a length in the order of a metre. This distance is as short as possible to reduce losses, avoid piping stagnation and limit the investment necessary to protect these elements from radiation.

In order to minimise the risks of biological development in the treatment unit 14 as well as in the final product, the inlet pipe 19 includes at least one filter. Thus, the collected urine is filtered on at least one filter preferably having pores of 0.22 μm. This helps retain urine epithelium cells, cell debris and bacteria typically found in the urine of a patient. This waste can be transferred to the secondary storage chamber 18.

The treatment unit 14 has a radiation-protective outer shell, in such a way that the interior of the treatment unit is radiation-protected for treatment. This protection is necessary to protect patients who have not been treated with internal vectorised radiotherapy, caregivers and accompanying people. This protection makes it possible to install the device 10 in any environment, in particular a non-radio-protected environment such as a normal hospital room.

As can be seen in FIG. 1, the treatment unit 14 includes:
at least one filtration and recovery device 20 specific to each radioactive isotope of interest,
a sorting module 22 connected to the urine collector 12 by the inlet pipe 19 and connected to the filtration and recovery device 20,
at least one primary storage chamber 24 in fluidic connection with each filtration and recovery device 20 configured to recover the corresponding radioactive isotope.

The sorting module 22 is thus configured to identify the radioactive isotope of interest in the collected urine and direct the collected urine to the at least one filtration and recovery device 20 corresponding to the detected radioactive isotope.

It is common to qualify a ‘radioactive isotope’ as ‘radioisotopes’.

For this purpose, in the embodiment shown in FIG. 1, the sorting module 22 includes a sorting chamber 26 housing a system 28 for detecting the radioactive isotope, for example a spectrometer 28. The sorting module 22 also includes a computing unit 30 connected to the detection system 28 (for example, a spectrometer) and to the pressure management system 16. The fluidic connection between the sorting chamber 26 of the sorting module 22 and the filtration and recovery device 20 is moreover provided by at least one anti-reflux valve 32, controlled by the computing unit 30 and configured to open only in response to the detection of the radioactive isotope of interest.

Upon arrival of the urine collected in the sorting chamber 26, the radioactive isotope of interest is thus detected by the detection system 28, for example by spectrometry (gamma/beta/alpha probe) of the type of radioisotope with differentiation, for example ¹³¹I, ¹⁷Lu, ²²⁵Ac, ¹⁶¹Tb, ¹⁴⁹Tb, ⁶⁷Cu.

In a manner known to any person skilled in the art, the molecules of radioactive isotopes (or radioisotopes) of interest are not injected alone to patients. What is injected into patients is an assembly of a plurality of molecules of which the radioisotope of interest. This assembly of molecules is conventionally called ‘radiopharmaceutical drug’ and is abbreviated to RPD. Thus, each RDP conventionally comprises, in most cases, a vector molecule and a radioactive isotope. By fixing on targets expressing a corresponding biomarker, RDP makes it possible to study a physiological process. The radioactive isotope makes it possible to track this molecule in the patient and makes it possible to carry out imaging examinations. In some special cases, patients are treated directly by the radioactive isotope of the RDP. This is then not an imaging method, but a means of treatment, directly. This way of binding the radioactive isotope of interest to a vector molecule is conventionally called internal vectorised radiotherapy. A chelator makes it possible to attach the radioactive isotope to the vector molecule. A free radioisotope is not attached to the vector molecule or to the chelator. The chelated radioisotope is a radioactive isotope attached by non-covalent bonds in the chelator.

Depending on the radioactive isotope detected, the computing unit 30 actuates the corresponding anti-reflux valve 32. Each anti-reflux valve 32 thus opens on an autonomous and isolated fluidic circuit forming, each, a filtration and recovery device 20.

Each filtration and recovery device 20 includes an ion retention element 36 and at least one reagent solution reservoir 38. Each filtration and recovery device 20 is configured to filter and recover the radioactive isotope of interest detected in the sorting chamber 26. Each ion retention element 36 is preferably single use.

Preferably, the ion retention element 36 is a cation exchange resin. This type of resin makes it possible to attach all positively charged molecules/ions to the solid phase and to allow neutral molecules or negatively charged molecules/ions to pass through. This type of resin must withstand a wide pH range and have a high affinity for divalent and/or trivalent ions while making the elution thereof possible afterwards in order to recover the free RDP/radioisotope. The resins of the Chromafix® PS-H⁺ type marketed by Macherey-Nagel™ could be adapted to this type of use.

Preferably, the reactive solution reservoir 38 contains a high-concentration cationic solution. It must be recharged regularly.

This ion retention element 36 makes it possible to ensure the elimination of water and to concentrate the radiopharmaceutical drug (RDP) and the radioactive isotope of interest. Indeed, urine being composed of more than 95% water, it is therefore critical to be able to eliminate excess water and concentrate the RDP/radioactive isotope of interest. More precisely, the ion retention element 36 makes it possible to trap free and chelated radioisotopes (RDP) (for example on a cation exchange resin). MRPs and radioisotopes are thus retained in the ion retention element 36 while excess water is discharged. The excess water (and generally the associated aqueous elements) thus recovered can be transferred, by the pressure management system 16, to the secondary storage chamber 18.

In the case where the collected urine does not include a radioactive isotope of interest, the collected urine can be transferred, by the pressure management system 16, directly into the secondary storage tank 18.

Once the excess water is removed, the radioisotopes are subsequently eluted with the high-concentration cationic solution from the reservoir 38. The trapping of free and chelated radioisotopes occurs when urine is circulated, by the pressure management system 16, through the ion retention element 36. The elution is also directed by the pressure management system 16. All this is controlled by the computing unit 30.

Upon exiting the filtration and recovery device 20, the radioactive isotope of interest recovered is transferred, by actuating the pressure management system 16, to the primary storage chamber 24 dedicated to storing the radioactive isotope of interest.

Each primary storage chamber 24 is adapted to the radioactive isotope selected by the sorting module 22 so as not to mix it with different radioisotopes.

As the ion retention element 36 of each filtration and recovery device 20 is single use, the sorting module 22 directs each new plant collected to a new filtration and recovery device 20, even if the radioactive isotope of interest is the same between two collections. However, all radioactive isotopes of interest of the same type are stored in the same primary storage chamber 24. To the extent possible of the capabilities of said primary storage chamber 24.

Each primary storage chamber 24 is thus connected to at least one, preferably a plurality of, filtration and recovery device(s) 20 by a pipe with at least one mechanical automatic closing and anti-reflux valve controlled by the computing unit 30.

Each primary storage chamber 24 is removable and replaceable for emptying the contents thereof. Each primary storage chamber 24 may be removable in a robotic and semi-automatic manner. In this case, the device 10 further includes a motorised carriage for transferring and changing each primary storage chamber 24 without handling.

Each primary storage chamber 24 preferably has a cube shape having a side length ranging from 40 to 50 cm. In such a way as to protect the environment from any form of radioactivity, each primary storage chamber 24 having an inner wall including a non-adherent material and cleanable in the autoclave. Each storage chamber 24 also has an outer wall at least partially made of material limiting the diffusion of gamma radiation. The thickness of these two walls is adapted to the emissions of each radioactive isotope of interest. Each primary storage chamber 24 further has a space between inner wall thereof and the outer wall thereof, this space being able to contain plexiglass in such a way as to limit the diffusion of β-radiation.

Each primary storage chamber 24 is connected to the control unit 30 of the sorting module 22 and has a connected gauge system making it possible to know in real time the amount of liquid or material accumulated.

The other radioisotopes not managed by the filtration and recovery device 20 can be transferred, by the pressure management system 16, to the secondary storage chamber 18.

To summarise, as illustrated in FIG. 2, the pressure management system 16 is particularly configured to generate:
a negative pressure in the sorting chamber 26 (and the secondary storage chamber 18) in response to the detection of liquid in the urine collector 12 (and/or stool collector 17) in order to transfer the collected urine (and stools) to the sorting chamber 26 (or the secondary storage chamber 18) (see reference 100 in FIG. 2),
a positive pressure in the sorting chamber 26 in response to the detection of the radioactive isotope of interest, in order to transfer the collected urine to the filtration and recovery device 20 (see reference 200 in FIG. 2),
a negative pressure in the primary storage chamber 24 in response to a release of the reactive solution from the reservoir 38 of the filtration and recovery device 20, in order to transfer the solutions to be retained to the primary storage chamber 24 (see reference 300 in FIG. 2).

When this is present, the pressure management system 16 maintains a permanent negative pressure in the secondary storage chamber 18 in order to send therein, as it progresses, the various waste of the various sorting, filtering and (according to embodiments) purification steps.

The device 10 according to the present invention therefore makes it possible to implement a method for recovering at least one radioactive isotope of interest present in the urine of a patient. The method includes the following steps of:
collecting the urine of the patient by means of the urine collector 12,
sucking the urine to the sorting module 22,
detecting the radioactive isotope of interest and the radioactive isotope of interest to a corresponding filtration and recovery device 20,
trapping the radioactive isotope of interest on the retention device 36 of the filtration and recovery device 20,
releasing the reactive solution in such a way as to release the radioactive isotope of interest,
fluidic transfer and storage of said radioactive isotope of interest to the primary storage chamber 24.

In some embodiments, the treatment unit 14 moreover includes a purification unit 40 configured to purify the radioisotopes recovered by the filtration and recovery device 20. In cases where the treatment unit 14 does not include a purification unit 40, the purification takes place outside of the device 10, either at the site for collecting the urine of the patient or at a conditioning site.

The purification unit 40 particularly includes a chelator column 42, an anti-chelator antibody column 44 (each column being preferably single use), a buffer solution reservoir 46, an acid solution reservoir 48 (each reservoir having to be filled regularly) for implementing a purification method as described below:
I. Passage over the chelator column 42 (for example a silica column grafted with chelators of the DOTA, or DTPA type, or any other chelator having a relative affinity for free radioisotopes and for releasing them under easy conditions). This step makes it possible to recover free radioisotopes in solution, such as, for example, [¹⁷⁷Lu]Lu⁺⁺⁺, [²²⁵Ac]Ac⁺⁺⁺, etc. (which are not chelated by the RDP). Anti-DOTA antibodies are quite specific to this chelator but may potentially recognise other types of macrocyclic chelators structurally similar to DOTA.
II. The flow through of the column in I. is then passed over the column of anti-chelator antibodies 44 (for example a resin column grafted with anti-chelator antibodies of the RDP (DOTA, etc.)) This step makes it possible to recover intact MRPs (or at least of the chelation part thereof containing the radioisotope).
III. If the device 10 includes a purification unit 40, the flow through of the column to II is sent directly into the secondary storage chamber 18. If not, the flow through of column II is not retained.
IV. The two columns in I. and II are thereafter washed with a weakly acidic solution (pH 6-6.5) from the buffer solution reservoir 46 in order to make it possible to detach the various molecules having non-specific interactions with the columns. If the device 10 includes a purification unit 40, it is sent directly into secondary storage chamber 18. If not, this solution is not retained.
V. The two columns in I. and II are thereafter treated with an acid solution (pH 3-5) from the acid solution reservoir 48 in order to make it possible to detach the free radioisotopes (column in I) and the entities retained on the column grafted with the chelator (column in II). This solution is retained and will subsequently be subjected to the chelator dissociation steps to find a solution of free radioisotopes only.

The column presented as an example in point I is specifically designed for the use of ¹⁷⁷Lu generators from ^{177m}Lu and has a significant advantage in the case where a large quantity of free radioisotopes is found in the collected urine (due to normal dissociation of the chelator or via DOTA radiolysis.

In some embodiments, the purification chamber 40 of the device 10 further includes a dissociation module 50 configured to dissociate the radio/isotope from the chelator. The solution recovered at point V is then either sent into a treatment centre in cases where the purification chamber 40 is not provided with the dissociation module, or transferred into the dissociation module to allow the dissociation between radioisotopes.

The dissociation module 50 makes it possible to implement a step of dissociating the radioisotope and the chelator.

This last step consists in forcing the radio-metal out of the chelator by acidifying the medium wherein the RDP is found. Indeed, in the presence of H⁺ ions, chelator/radioisotope dissociation may occur more easily. It is combined with the heating of the highly concentrated H⁺ solution for accelerating chelator/radioisotope dissociation.

It should be noted that the chelator/metal dissociation is a very slow step and one of the advantages of the present invention, when the device 10 is provided with a purification chamber 40 including a dissociation module 50 is to accelerate this process in order to reuse a large part of the free radioisotope for new RDP markings.

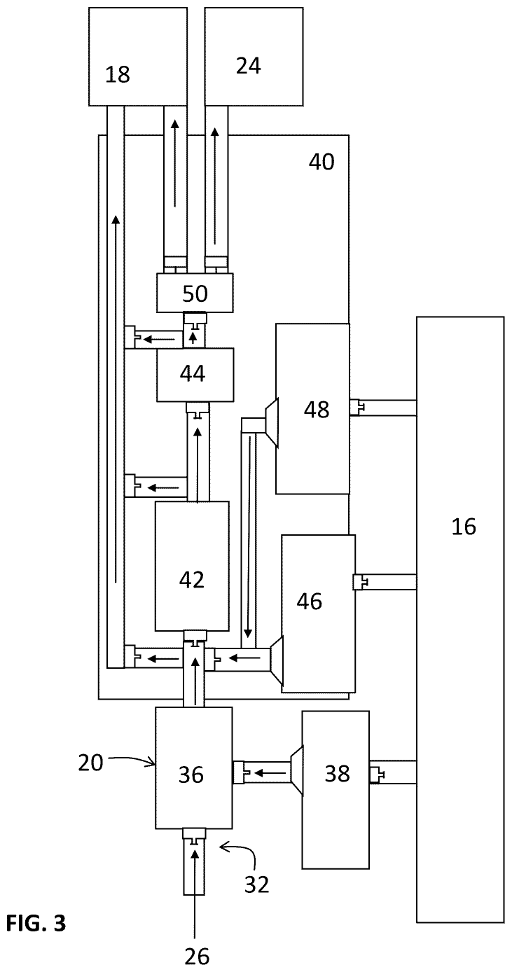

As can be seen in FIG. 3, each element of the treatment unit 14 is preferably connected, by a system of anti-reflux valves and fluidic connections, to the secondary storage chamber 18 (when this is present) in order to be able to evacuate the waste at each step of the radioisotope recovery process. The presence of anti-reflux valves is important to ensure that fluids flow only in one direction, in the direction of the secondary storage chamber 18 (or the primary storage chamber 24, if applicable).

In the context of a patient treated simultaneously with a plurality of radioisotopes of interest having a half-time for envisaging separation and reuse thereafter, the device 10 according to the present invention could comprise an isotope separation module (not shown) for implementing a separation method resulting from the different radioactive isotopes of interest of the steps of ‘filtration and recovery of radioactive isotopes’. This separation method may consist of a chromatographic separation.

Thus, the present invention presents an integrated solution for recovering waste produced by patients injected with IVR treatments such as urine and stool. Thus, the present invention makes it possible to revalorise urine in order to extract the various radioactive molecules and the attached isotopes thereof. Stools are collected for subsequent treatment in the case of isotopes that are mostly evacuated by the stools or decay and elimination.

The various steps of the device 10 according to the present invention integrate all of the steps ranging from the collection of radioactive urine to the reconditioning of the radioisotope for good manufacturing practice (GMP) reconditioning.

1-10. (canceled)

11. A device for recovering at least one radioactive isotope of interest present in the urine of a patient, the device being intended to be connected to toilets, the device comprising a urine collector, a treatment unit, a pressure management system configured to move urine from the urine collector to and within the treatment unit, the treatment unit including:
at least one filtration and recovery device specific to each radioactive isotope of interest, including an ion retention element and at least one reactive solution reservoir, the filtration and recovery device being configured to filter and recover the radioactive isotope of interest,
a sorting module connected to the urine collector configured to identify the radioactive isotope of interest in the collected urine and direct the collected urine to the at least one filtration and recovery device corresponding to the detected radioactive isotope,
at least one primary storage chamber in fluidic connection with each filtration and recovery device configured to recover the corresponding radioactive isotope.

12. The device according to claim 11, wherein the sorting module is provided with a detection system configured to detect the radioactive isotope of interest.

13. The device according claim 11, further comprising: a primary storage chamber for each radioactive isotope of interest.

14. The device according to claim 11, wherein the suction the urine of the patient is activated by detecting liquid in the urine collector.

15. The device according to claim 11, further comprising: a secondary storage chamber configured to recover aqueous elements of the collected urine, said secondary storage chamber being in fluidic connection with the sorting module and each filtration and recovery device.

16. The device according to claim 11, wherein the pressure management system is configured to generate:
a negative pressure in the sorting module in response to the detection of liquid in the urine collector,
a positive pressure in the sorting module in response to the detection of the radioactive isotope of interest.
a negative pressure in the primary storage chamber in response to a release of the reactive solution from the reservoir of the filtration and recovery device.

17. The device according to claim 11, wherein the ion retention element of the filtration and recovery device is a cation exchange resin.

18. The device according to claim 11, further comprising: a stool collector.

19. The device according to claim 11, further comprising: a purification chamber.

20. A method for recovering at least one radioactive isotope of interest present in the urine of a patient implemented by the device according to claim 11, the method includes the steps of:
collecting the urine of the patient by the urine collector,
sucking the urine to the sorting module,
detecting the radioactive isotope of interest and the radioactive isotope of interest to a corresponding filtration and recovery device,
trapping the radioactive isotope of interest on the retention device of the filtration and recovery device,
releasing the reactive solution in such a way as to release the radioactive isotope of interest,
fluidically transferring and storing said radioactive isotope of interest to/in the primary storage chamber.
